(19) United States
(12) Patent Application Publication
(10) Pub. No.: US 2011/0082448 A1
(43) Pub. Date: Apr. 7, 2011

(54) Pharmaceutical Compositions

(76) Inventor: Alfredo Emilio BRUNO-RAIMONDI, Biel-Benken (CH)

(21) Appl. No.: 12/893,295

(22) Filed: Sep. 29, 2010

Related U.S. Application Data
(63) Continuation of application No. 11/483,689, filed on Jul. 10, 2006.
Application No. 11/483,689 is a division of application No. 10/343,801, filed on Mar. 14, 2003, now abandoned.

(30) Foreign Application Priority Data
Aug. 4, 2000 (GB) 0019283.1

(51) Int. Cl. A61B 18/20

(52) U.S. Cl. 606/10

(57) ABSTRACT

A method for the topical treatment of nail diseases, e.g. onychomycosis, after preparation of the infected nail with a laser beam.

### FIELD OF THE INVENTION

The present invention relates to a method for the topical treatment of nail diseases, e.g. onychomycosis, after preparation of the infected nail with e.g. a pulsed laser beam.

### BACKGROUND OF THE INVENTION

Diseases of the nail, such as onychomycosis, atopic eczema, or psoriasis of the nail are difficult to treat. Although for some of these diseases effective treatment by the systemic, e.g. oral, route is available, there is still the need for an effective topical method of treatment.

Onychomycosis accounts for up to 50% of all nail diseases and affects 2% to 18% or more of the world's population. Some studies suggest that up to 48% of the population may be affected by age 70. Toenail infection is several times more common than fingernail infection and is more difficult to treat because of its slower growth rate.

Terbinafine is an orally effective anti-fungal agent, available under the registered trademark Lamisil. It is effective in a wide range of fungal infections. Terbinafine is particularly useful against dermatophytes, contagious fungi that invade dead tissues of the skin or its appendages such as stratum corneum, nails, and hair. The effects of these fungi on the nails may be unsightly, seriously complicate foot-care, have a deleterious impact on patients' overall quality of life, and well-being and impair the patients' ability to work. If left untreated, the fungi can deform toe-nails permanently and lead to pain on walking. Additionally the fungi can lead to fissures in the skin encouraging bacterial infections. Serious complications as a result of these infections may occur in people suffering from diabetes such as diabetic foot syndrome including primary disease-related complications, e.g. gangrene, that, ultimately, can be life-threatening or require amputations. Other high-risk patient sub-groups include patients infected with human immunodeficiency virus (HIV), patients with acquired immunodeficiency syndrome (AIDS), and patients with other types of immunosuppression (e.g. transplant recipients and patients on long-term corticosteroid therapy). Diagnosis is confirmed by demonstrating the pathogenic fungus in scrapings of the lesions either by microscopic examination or by culture.

For the onychomycosis use, an antifungal, e.g. terbinafine, is normally orally administered as an immediate release tablet. Terbinafine treatment is typically required over 12 weeks. The progress of its clinical effectiveness is seen with growth of the healthy nail, pushing out and replacing, the diseased unsightly nail containing debris and dead fungus. About 10 months is taken for a totally new toe-nail to form.

Whereas e.g. terbinafine is highly active upon oral administration, systemic treatment of onychomycosis offers some disadvantages, e.g. exposure of the whole organism to the drug substance and the need for rather high doses. Therefore the possibility of local, namely topical treatment is highly desirable and would be preferred by many patients. However, one difficulty in the topical treatment of onychomycosis or other nail diseases may be related to insufficient penetration of the drugs into deeper layers of the nail and nail bed.

The nail plate is formed by layers of keratinized cells produced by the nail matrix, a highly proliferative epidermal tissue. Besides keratin, the rigid structure of the nail contains numerous trace minerals including calcium. The nail plate overlays the nail bed, a noncornified tissue. At the interface, nail bed cells are carried distally by the nail plate during the growth towards the free margin. The keratinization of the nail plate in the matrix occurs both on the dorsal and ventral side of the forming nail plate. There are at least two discernible macroscopic strata, with possible a third. These are the dorsal nail plate and the intermediate nail plate with the third under-layer or ventral plate contributed by the cells of the lunula. The dorsal plate is harder laminated thus more compact and making the penetration of compounds more difficult.

The hydrated nail behaves as a hydrogel of high ionic strength forming a thick hydrophilic barrier making it extremely difficult for hydrophobic drugs to penetrate the nail plate down to the nail bed. The thickness of the nail and its relatively compact structure make it even more difficult for topically applied drugs, e.g. antifungal agents, to penetrate the nail.

### SUMMARY OF THE INVENTION

According to the present invention it has now been found that onychomycosis can be successfully treated by forming one or more small orifices, e.g. traversing the entire nail plate or etching the nail plate, and administering e.g. an antifungal-, e.g. a terbinafine-, containing composition to the nail. Preferably, the orifices may have a size (diameter) of e.g. 10 μm (microns) to 2 mm, e.g. 50 μm (microns) to 1 mm, e.g. 140 μm (microns) to 1 mm.

Accordingly, in one aspect the present invention provides a method for the treatment of onychomycosis which method comprises forming one or more orifices in the nail and administering e.g. an antifungal-containing, e.g. preferably a terbinafine-, containing, pharmaceutical composition to the nail.

### BRIEF DESCRIPTION OF THE DRAWINGS

FIG. 1 shows an exemplary view of an orifice array; and

FIG. 2 shows an exemplary device for using the methods of the present invention.

### DETAILED DESCRIPTION OF THE INVENTION

“Orifice” as herein described means any small hole or depression that penetrates 80 to 100% of the nail plate, preferably 90 to 99%.

To date, orifices in finger- or toenails have been produced with a mechanical drill, e.g. as described in U.S. Pat. No. 4,180,058, or with a heated wire for burning a hole. In U.S. Pat. No. 5,947,956, a laser apparatus for making holes and etchings is described.

As antifungal, allyl amines such as terbinafine or naftifine, benzylamines such as butenafine, and/or azole-based antifungals such as tioconazole, econazole, oxiconazole, itroconazole, fluconazole, ketoconazole, miconazole and clotrimazole may be used. Allyl amines and benzyl amines may be in free base form or acid addition salt form, preferably in form of hydrochloride salt.

The antifungal-containing pharmaceutical composition may comprise a highly concentrated antifungal formulation from about 10 to about 100% of antifungal by weight of the composition, e.g. more than about 70%, or e.g. about 100%, e.g. substantially pure antifungal, e.g. terbinafine, powder.

Alternatively, the antifungal-containing pharmaceutical composition comprises antifungal from about 1 to about 10% by weight of the composition.

The antifungal-containing pharmaceutical composition may be e.g. liquid, viscous or semi-solid, e.g. in form of a cream, a gel, a solution, a lotion, an ointment, a patch or a nail varnish; the formulation may e.g. be a liposomal preparation. If the composition is viscous, it may be warmed up before pouring it in the orifice, e.g. to facilitate penetration. Alternatively, the antifungal-containing pharmaceutical composition may be e.g. solid, e.g. a powder.

The preferred type and strength of formulation may be chosen according to e.g. the degree of infection of nail and/or size of holes. Furthermore, a surfactant may be added to the antifungal formulation, e.g. to facilitate its penetration into and through the orifices.

In another aspect, the present invention provides for the use of an antifungal, e.g. terbinafine, to produce a medicament to be administered to penetrate effectively an orifice of a nail.

Before administering the antifungal-containing pharmaceutical composition, the nail, e.g. after being prepared with the orifices, may be treated with a surfactant e.g. such as i) natural products, e.g. Aloe Vera, e.g. in form of a gel, or ii) a non-ionic surfactant which is inert, non-irritant, and does not have a pharmaceutical response, e.g. in order to facilitate penetration of the anti-fungal formulation.

Immediately after administering the antifungal-, e.g. terbinafine-, containing pharmaceutical composition to the nail, a, e.g. protective, e.g. inert, layer comprising e.g. a nail varnish, porcelain layer, an artificial nail or a polymer foil, e.g. a patch, may be applied onto the nail. This may ensure that the pharmaceutical composition remains in the nail and may also prevent bacteria and dirt reaching the nail bed. The protective layer preferably also comprises an anti-fungal. Application of e.g. a colored nail varnish may mask the unpleasant appearance of the infected nail.

In a further aspect the present invention provides a method as described above which method further comprises administering a, e.g. protective, layer onto the nail.

As the nail grows, the small orifice typically closes, thus trapping the antifungal into the nail plate. Accordingly, after some time, e.g. a few days to one week, application of a protective layer may no longer be needed.

The present applicants have found that the orifice in the nail may be easily formed in a fraction of a second by a device which comprises a laser, e.g. an Erbium (Er:YAG) laser, Neodym (Nd:YAG) laser, OPO laser, Holmium (Ho:YAG), a nitrogen laser or CO₂ laser where YAG stands for yitrium aluminium garnet.

In another aspect, the present invention provides a method for the treatment of a nail infected with onychomycosis which method comprises forming one or more orifices in the nail with a device comprising a laser, e.g. an Erbium (Er:YAG) laser, Holmium (Ho:YAG) or nitrogen laser, and administering an antifungal-containing pharmaceutical composition to the nail.

The use of a laser-based device to form an orifice in the nail according to the present invention is especially advantageous because of e.g. high precision, high speed, lack of or little pain, and no risk of bleeding or of secondary infections.

Laser surgery, i.e. the method of the present invention to perforate the nail, is based on the photo-ablation process which refers to the melting and explosion of hard tissues. Pulsed laser irradiation of a selected wavelength, power and pulse duration according to the thermal, mechanical and spectral characteristics of the tissue of interest, achieve photo-ablation. The irradiation of a tissue with a pulsed laser of low to moderate power density induces thermal changes through absorption with minor mechanical and reversible changes. However, at much higher power and when the confinement of a given energy in a defined volume during a short time satisfies the threshold conditions, hard tissue can be selectively destructed, e.g. for drilling or cutting purposes. The deposited electromagnetic energy is almost entirely transformed into mechanical energy (i.e. hν≈mv²/2) and the illuminated region is ejected in the form of debris escaping the orifice at ca. 1'000 m/s. In a “clean”, i.e. efficient, photoablation process, as the debris removes most of the deposited energy, the irradiated tissue, e.g. the tissue in contact with the nail bed, is not heated thus reducing the causes for pain.

Although the nail bed is known to be very sensitive, according to the present invention it has been shown that nail diseases can be treated using a laser based technology with minimal patient discomfort. It has been found that the associated pain is minimal and can be tolerated without anesthetics.

As the ablation temperature is e.g. higher than about 100° C., disinfection of nail and/or nail orifices may not be required. Therefore, the present invention provides a method as described above which method does not involve a disinfection step.

Because the spectral characteristics of human tissues in the NIR region are dominated by the spectrum of water, the lasers that can be used for tissue micromachining are the CO₂ gas laser lasing at λ=10.6 μm, and the two most recently developed solid state lasers, the Ho:YAG and Er:YAG delivering light (electromagnetic radiation) at λ=2.1 μm and λ=2.94 μm, respectively. A Nd:YAG laser or an OPO laser may also be used. According to the present invention, the Ho:YAG or Er:YAG laser are preferred.

According to the present invention it has been found that one or more small orifices may be formed with a, preferably tightly focussed, single laser shot of only less than ca. 250 mJ of power, e.g. 50 mJ, ca. 250 μs of duration at a repetition rate of 3 Hz for a healthy nail of about 0.7 mm thickness. Although we do not want to be bound by any theory, it is believed that the surprising much easier to achieve photoablation conditions in the nail, as compared to bone or teeth, appears to berelated to its structure. As mentioned above, the nail behaves as a hydrogel. Consequently its spectrum is dominated by the absorption of water and, the melting point of humidified keratin should be at a much lower temperature than that of hydroxylapatite or enamel contained in bone or teeth, respectively. For this reason, typically the orifices are substantially circular, conical, frusto-conical, hemi-spherical or cylindrical in cross-section, preferably cylindrical, mimicking the stark focussed laser beam profile. As the process occurs faster than the thermal diffusion rate in nail, no damage in the orifices in the form of craters are observed. Most importantly, the orifices are done with a single shot or a few weak laser shots thus reducing the heating of the nail, a source of pain, to a minimum. This aspect is also time-relevant if one wishes to make arrays of orifices as explained below. With a more refined optical set-up, using e.g. masks, the orifices may be made much smaller or, with the use of Diffractive Optical Elements (DOEs), such as a Dammann grating arrays of orifices can be done simultaneously.

According to the present invention, the nail can be used as a natural depot, e.g. reservoir, wherein an array of equally spaced laser formed orifices in the nail is made and subsequently filled with the antifungal-containing composition to be released, e.g. slowly released, into the nail bed. Because it takes the nail 5 to 10 months to regenerate, the orifices constitute an ideal depot, e.g. reservoir, for the slow delivery of antifungal, e.g. terbinafine, into the nail bed for the topical treatment of onychomycosis.

The laser-based device, e.g. laser light delivery device, for forming one or more orifices in one or more nails may comprise the following elements:

(a) a laser capable of inducing ablation on the nail plate, i.e. for perforating nail, e.g. making orifices, e.g. as main photoablation laser, e.g. a pulsed, e.g. solid state, laser light source, e.g. Erbium (Er:YAG) laser (λ=2.94 microns), a Nd:YAG laser, an OPO laser, a Ho:YAG laser (λ=2.1 microns), or a CO₂ laser (λ=10.6 microns), preferably an Erbium (Er:YAG) or Holmium (Ho:YAG) laser
(b) supporting means to fix/position, e.g. by a clamp, the toe or finger to be treated, leaving the nail plate uncovered for the laser illumination or exposure,
(c) preferably a second visible continuous wave (cw) laser, a so-called pointing or targeting laser (acting as a pointer) of e.g. low power, e.g. a laser emitting in the visible region, e.g. a red He—Ne lasing at λ=633 nm, or e.g. a laser diode, may be used to ensure a higher micromachining precision, i.e. visualisation of the spots to be perforated,
(d) a computer controlled xyz translation stage module to position the laser beam in the desired area of the nail preferably a so-called laser beam scanner. Alternatively the support (b) may be mounted on this translation stage so that the laser beam is fixed and the toe or finger moves. Alternatively, the toe or finger may be fixed and the laser beam is mounted on the translation stage to move on the nail plate.
(e) means for directing laser beams, e.g. a mirror, e.g. a dichroic mirror, or a prism, to co-axially mix the laser beams from the ablation laser (a) and optionally the pointing laser (d),
(f) means to remove the nail debris, e.g. a nail debris removing disk, e.g. vacuum system or a flat thin piece of, e.g. round, material which is transparent to the laser beam wavelengths, e.g. a round disk made of quartz, e.g. fused silica, may be used to prevent dirtying of the focussing optics elements (i),
(g) means to clean the nail debris removing disk (f) from the nail debris, e.g. a thin tube ejecting a jet of sterilized water,
(h) means to ensure that the nail debris removing disk (f) is clean when the ablation laser is fired, e.g. a wiper, e.g. made of a flexible material, e.g. rubber or silicon polymer, or a brush,
(i) one or more focussing optics elements, e.g. manual or in an auto-focus mode, comprising one or more lenses placed between the lasers (a) and (d) and the nail debris removing disk (f),
(j) computing means, e.g. a personal computer, serving e.g. the following purposes: to monitor the nail plate by means of a video camera or charged coupled device camera (k), to place the laser beams (a) and (d) to the points of the nail plate where orifices are to be formed by means of a computer controlled xyz translation stage (c), to control and/or select the different laser parameters, e.g. the firing of the laser when the desired position of the xyz translation stage (c) has been reached, or the laser power, the pulse duration, or wavelength, e.g. if the laser is a tunable laser,
(k) means for monitoring the nail plate, e.g. a video camera or charged coupled device camera, to monitor the nail plate on the screen of computing means, e.g. a personal computer (j), preferably a charged coupled device camera,
(l) feedback means, e.g. a sensor, e.g. a photoacoustic sensor made of a piezoelectric (PZE) material, to ensure that the laser stops after the orifice has reached a predetermined depth, e.g. the nail bed. Preferably the ablation laser (a) conditions are chosen to form an orifice with a single shot without reaching soft tissue. The feedback sensor may be used to choose and optimize this condition starting from the first orifice formed,
(m) an optical element may be used to multiplex the laser beams (a) and (d), e.g. a diffractive optical element, e.g. a Dammann grating, to make more than one orifice, e.g. an array of equally spaced orifices, by a single shot thereby avoiding to make the orifices one-by-one in a subsequent mode.

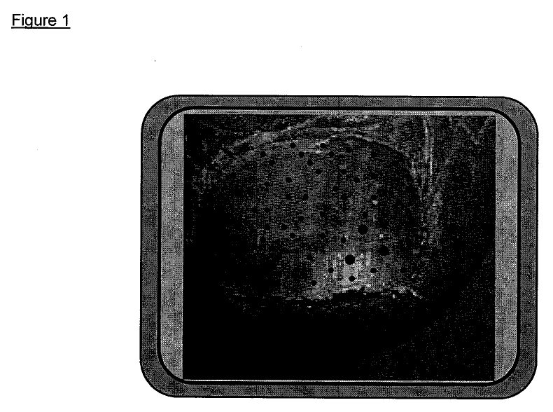

Preferably, the operator uses a software that makes use of the image of the captured and stored nail image to help the operator define the pattern, size, geometry etc. of the orifice-array and, with this information, calculate the individual orifice coordinates with respect to the nail, compute corresponding laser ablation conditions, and/or run the positioning elements, e.g. run the laser or lasers and the hardware. FIG. 1 shows an example of an orifice array.

Preferably, the laser light delivery device, e.g. especially the support (b), the mirror (e) and the device to remove the nail debris (f), is easy to clean and may be run under sterile conditions.

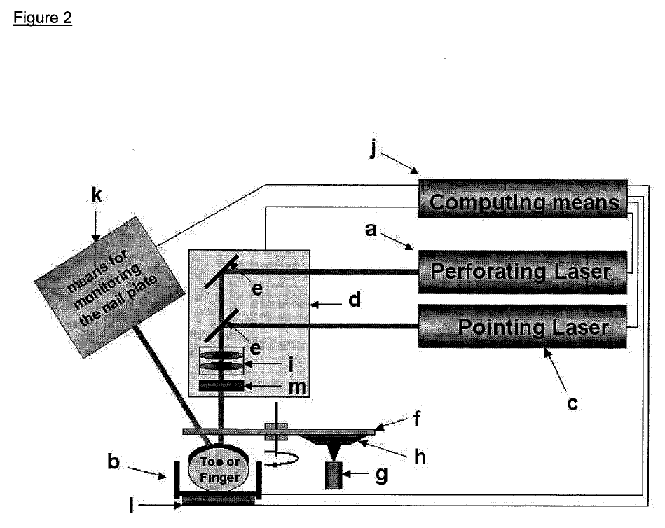

Accordingly, in a further aspect the present invention provides a device, e.g. for the use in a method according to the present invention, e.g. as described in FIG. 2, which device comprises

(a) a laser for perforating nail and optionally one or more of the following elements
(b) supporting means to fix/position the toe or finger to be treated,
(c) a pointing laser,
(d) a xyz translation stage module,
(e) means for directing laser beams,
(f) means to remove the nail debris,
(g) means to clean the nail debris removing disk (f) from the nail debris,
(h) means to ensure that the nail debris removing disk (f) is clean when the ablation laser is fired,
(i) one or more focussing optics comprising one or more lenses,
(j) computing means,
(k) means for monitoring the nail plate (l) feedback means,
(m) a diffractive optical element,
(n) software to define the pattern, size, geometry etc. of the orifice-array, to calculate the individual orifice coordinates, to compute corresponding laser ablation conditions, and/or to run the positioning elements.

Preferably, the device, e.g. for the use in a method according to the present invention comprises the following elements:

(a) a laser for perforating nail, e.g. for making orifices, e.g. a solid state pulsed laser light source, e.g. Erbium (Er:YAG) laser, Neodym (Nd:YAG) laser, OPO laser, Holmium (Ho:YAG), or CO₂ laser, preferably Erbium (Er:YAG) laser, Holmium (Ho:YAG), or CO₂ laser, even more preferably an Erbium (Er:YAG) laser, e.g. as main photoablation laser,
(b) supporting means to fix/position the toe or finger to be treated,
(c) a pointing laser, e.g. a visible continuous wave (cw) laser of e.g. low power, from e.g. a laser emitting in the visible region, e.g. a red He—Ne laser or a laser diode, and
(d) optionally a xyz translation stage module.

The following Examples illustrate the invention.

### Example 1

A series of orifices (ca. 20 orifices) are drilled in a hydrated in-vitro 0.7 mm thick human nail using a Er:YAG laser. The diameter of the orifices ranges from 1 mm to 140 microns.

### Example 2

A series of orifices (ca. 20 orifices) are drilled in vivo without anesthetics in various nails from a healthy subject using a Er:YAG laser. The diameter of the orifices ranges from 1 mm to 50 microns.

### Example 3

A composition containing 10% terbinafine based on the total weight of the composition is prepared and administered to a laser-perforated nail of a patient suffering from onychomycosis once daily for 12 weeks. The progress of clinical effectiveness is seen with growth of the healthy nail.

1. A method for the treatment of a nail infected with onychomycosis which method comprises forming one or more orifices in the nail with a device comprising an Erbium (Er:YAG) laser or Holmium (Ho:YAG) laser and administering an antifungal-containing pharmaceutical composition to the nail.

2. A method according to claim 1 wherein the antifungal-containing pharmaceutical composition is liquid, viscous or semi-solid.

3. A method according to claim 1 or 2 wherein the pharmaceutical composition comprises the antifungal in an amount of from 1 to 10% by weight of the composition.

4. A method according to claim 1 or 2 wherein the pharmaceutical composition comprises the antifungal in an amount of from 10 to 100% by weight of the composition.

5. A method according to claim 4 wherein the pharmaceutical composition comprises the antifungal in an amount of more than about 70% by weight of the composition.

6. A method according to claim 1 wherein the pharmaceutical composition comprises substantially pure antifungal powder.

7. A method according to any preceding claim which further comprises treatment of the nail with a surfactant before administering the antifungal-containing composition.

8. A method according to any preceding claim which method further comprises administering a protective layer onto the nail.

9. Use of an antifungal, e.g. terbinafine, to produce a medicament to be administered to penetrate effectively an orifice of a nail.

10. A method or use according to any preceding claim wherein the antifungal is an allyl amine such as terbinafine or naftifine, a benzylamine such as butenafine, and/or an azole-based antifungal such as tioconazole, econazole, oxiconazole, itroconazole, fluconazole, ketoconazole, miconazole and clotrimazole.

11. A method or use according to any preceding claim wherein the antifungal is terbinafine.

12. A terbinafine-containing pharmaceutical composition suitable for the use in a method according to any one of claims 1 to 8.

13. A device comprising
(a) a laser for perforating nail
(b) supporting means to fix/position the toe or finger to be treated,
(c) a pointing laser, and
(d) optionally a xyz translation stage module.

14. A device according to claim 13, wherein the perforating laser is an Erbium (Er:YAG) or
Holmium (Ho:YAG) laser.

15. A device according to claim 13 or 14, wherein the pointing laser is a visible continuous wave (cw) laser e.g. a red He—Ne laser or a laser diode.

16. A device according to any one of claims 13 to 15 further comprising means for monitoring the nail plate.
